(19) United States
(12) Patent Application Publication
(10) Pub. No.: US 2023/0215059 A1
(43) Pub. Date: Jul. 6, 2023

(54) THREE-DIMENSIONAL MODEL RECONSTRUCTION

(71) Applicant: Auris Health, Inc., Redwood City, CA (US)

(72) Inventors: Elif AYVALI, Redwood City, CA (US); Bulat Ibragimov, Coppenhagen (DK)

(73) Assignee: Auris Health, Inc., Redwood City, CA (US)

(21) Appl. No.: 18/067,698

(22) Filed: Dec. 16, 2022

Related U.S. Application Data
(60) Provisional application No. 63/295,518, filed on Dec. 31, 2021.

(51) Int. Cl. G06T 11/00; G06T 15/08; G06V 10/82; G06V 10/774; G06V 40/10

(52) U.S. Cl. CPC G06T 11/008; G06T 15/08; G06V 10/82; G06V 10/7753; G06V 40/10; G06T 2210/41

(57) ABSTRACT

The present disclosure relates to systems, devices, and methods to reconstruct a three-dimensional model of an anatomy using two-dimensional images.

### RELATED APPLICATION(S)

This application claims priority to U.S. Provisional Application No. 63/295,518, filed Dec. 31, 2021, entitled THREE-DIMENSIONAL MODEL RECONSTRUCTION, the disclosure of which is hereby incorporated by reference in its entirety.

### BACKGROUND

Various medical procedures involve the use of one or more devices configured to penetrate the human anatomy to reach a treatment site. Certain operational processes can involve localizing a medical instrument within the patient and visualizing an area of interest within the patient. To do so, many medical instruments may include sensors to track the location of the instrument and may include vision capabilities, such as embedded cameras or the compatible use with vision probes.

### BRIEF DESCRIPTION OF THE DRAWINGS

Various embodiments are depicted in the accompanying drawings for illustrative purposes and should in no way be interpreted as limiting the scope of the disclosure. In addition, various features of different disclosed embodiments can be combined to form additional embodiments, which are part of this disclosure. Throughout the drawings, reference numbers may be reused to indicate correspondence between reference elements.

FIG. 1 is a block diagram that illustrates an example medical system for performing various medical procedures in accordance with aspects of the present disclosure.

FIG. 2 is a diagram illustrating a reconstructed three-dimensional model in accordance with one or more embodiments.

FIG. 3 is a block diagram illustrating an example dataflow of the model reconstruction module of FIG. 1, according to an example embodiment.

FIG. 4 is a flow-chart illustrating a method to reconstruct a three-dimensional model from two-dimensional images acquired during or as part medical procedure, according to an example embodiment.

FIG. 5 is a system diagram illustrating a neural network generation system, according to an example embodiment.

FIG. 6 is a flowchart illustrating a method to generate a trained neural network usable to reconstruct a three-dimensional model from a set of two or more two-dimensional images, according to an example embodiment.

FIG. 7 is a system diagram illustrating a personalized neural network generation system, according to an example embodiment.

FIG. 8 is a diagram illustrating a reconstructed three-dimensional model 800, according to an example embodiment.

### DETAILED DESCRIPTION

The headings provided herein are for convenience only and do not necessarily affect the scope or meaning of disclosure. Although certain exemplary embodiments are disclosed below, the subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Overview

The present disclosure relates to systems, devices, and methods to generate three-dimensional models of an anatomy. Many medical procedures rely on accurate representations of a patient's anatomy and guidance in controlling instruments within that anatomy. For example, in kidney stone removal through a percutaneous access, accurate and safe stone removal may depend on the selection of a calyx in which the percutaneous instrument enters the kidney. The selected calyx should offer not only easy access to the stone but also ensure that the needle trajectory from a patient's skin to the kidney collective system agrees with the patient's anatomy. The selection of the calyx can involve analysis of pre-operative computed tomography (CT) images and intra-operative fluoroscopic images. A pre-operative CT image is often acquired without contrast agent and can therefore only visualize the stone location against the kidney volume and is not sufficient for visualizing the kidney collective system morphology. An intra-operative fluoroscopic image, which may have a comparatively higher resolution, may be acquired with contrast agent, which reveals the relative locations of kidney calyces. However, fluoroscopic images are two-dimensional and understanding the volumetric kidney morphology from single/multiple fluoros can be challenging.

Embodiments described herein may reconstruct three-dimensional models of anatomy from a limited set of two-dimensional images.

Three-Dimensional Pose Estimation System

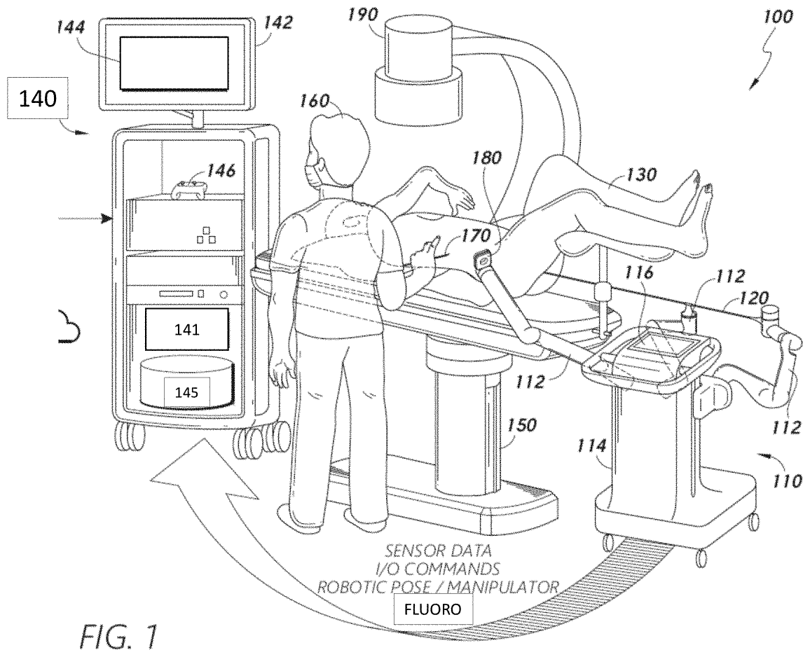

FIG. 1 is a block diagram that illustrates an example medical system 100 for performing various medical procedures in accordance with aspects of the present disclosure. The medical system 100 includes a robotic system 110 configured to engage with and/or control a medical instrument 120 to perform a procedure on a patient 130. The medical system 100 also includes a control system 140 configured to interface with the robotic system 110, provide information regarding the procedure, and/or perform a variety of other operations. For example, the control system 140 can include a display(s) 142 to present certain information to assist the physician 160. The display(s) 142 may be a monitor, screen, television, virtual reality hardware, augmented reality hardware, three-dimensional imaging devices (e.g., hologram devices) and the like, or combinations thereof. The medical system 100 can include a table 150 configured to hold the patient 130. The system 100 can further include an electromagnetic (EM) field generator 180, which can be held by one or more robotic arms 112 of the robotic system 110 or can be a stand-alone device. In examples, the medical system 100 can also include an imaging device 190 which can be integrated into a C-arm and/or configured to provide imaging during a procedure, such as for a fluoroscopy-type procedure.

In some implementations, the medical system 100 can be used to perform a percutaneous procedure. For example, if the patient 130 has a kidney stone that is too large to be removed through a urinary tract, the physician 160 can perform a procedure to remove the kidney stone through a percutaneous access point on the patient 130. To illustrate, the physician 160 can interact with the control system 140 to control the robotic system 110 to advance and navigate the medical instrument 120 (e.g., a scope) from the urethra, through the bladder, up the ureter, and into the kidney where the stone is located. The control system 140 can provide information via the display(s) 142 regarding the medical instrument 120 to assist the physician 160 in navigating the medical instrument 120, such as real-time images captured therewith.

Once at the site of the kidney stone (e.g., within a calyx of the kidney), the medical instrument 120 can be used to designate/tag a target location for the medical instrument 170 (e.g., a needle) to access the kidney percutaneously (e.g., a desired point to access the kidney). To minimize damage to the kidney and/or the surrounding anatomy, the physician 160 can designate a particular papilla as the target location for entering into the kidney with the medical instrument 170. However, other target locations can be designated or determined. To assist the physician in driving the medical instrument 170 into the patient 130 through the particular papilla, the control system 140 can provide a visualization interface 144, which can include a rendering of a three-dimensional model of an anatomy generated based on two-dimensional images captured by the system 100, such as a fluoroscopic image. As is explained in greater detail, the visualization interface 144 may provide information to the operator that is helpful in driving the medical instrument 170 to the target location.

Once the instrument 170 has reached the target, the physician 160 can use the medical instrument 170 and/or another medical instrument to extract the kidney stone from the patient 130. One such instrument may be a percutaneous catheter. The percutaneous catheter may be an instrument with steering capabilities, much like the instrument 120, but may, in some embodiments, lack a dedicated camera or location sensor. Some embodiments may use the augmented visualization interface 144 to render augmented images that are helpful in driving the percutaneous catheter within the anatomy.

Although the above percutaneous procedure and/or other procedures are discussed in the context of using the medical instrument 120, in some implementations a percutaneous procedure can be performed without the assistance of the medical instrument 120. Further, the medical system 100 can be used to perform a variety of other procedures.

Moreover, although many embodiments describe the physician 160 using the medical instrument 170, the medical instrument 170 can alternatively be used by a component of the medical system 100. For example, the medical instrument 170 can be held/manipulated by the robotic system 110 (e.g., the one or more robotic arms 112) and the techniques discussed herein can be implemented to control the robotic system 110 to insert the medical instrument 170 with the appropriate pose (or aspect of a pose, such as orientation or position) to reach a target location.

In the example of FIG. 1, the medical instrument 120 is implemented as a scope and the medical instrument 170 is implemented as a needle. Thus, for ease of discussion, the medical instrument 120 is referred to as “the scope 120” or “the lumen-based medical instrument 120,” and the medical instrument 170 is referred to as “the needle 170” or “the percutaneous medical instrument 170.” However, the medical instrument 120 and the medical instrument 170 can each be implemented as a suitable type of medical instrument including, for example, a scope (sometimes referred to as an “endoscope”), a needle, a catheter, a guidewire, a lithotripter, a basket retrieval device, forceps, a vacuum, a needle, a scalpel, an imaging probe, jaws, scissors, graspers, needle holder, micro dissector, staple applier, tacker, suction/irrigation tool, clip applier, and so on. In some embodiments, a medical instrument is a steerable device, while other embodiments a medical instrument is a non-steerable device. In some embodiments, a surgical tool refers to a device that is configured to puncture or to be inserted through the human anatomy, such as a needle, a scalpel, a guidewire, and so on. However, a surgical tool can refer to other types of medical instruments.

In some embodiments, a medical instrument, such as the scope 120 and/or the needle 170, includes a sensor that is configured to generate sensor data, which can be sent to another device. In examples, sensor data can indicate a location/orientation of the medical instrument and/or can be used to determine a location/orientation of the medical instrument. For instance, a sensor can include an electromagnetic (EM) sensor with a coil of conductive material. Here, an EM field generator, such as the EM field generator 180, can provide an EM field that is detected by the EM sensor on the medical instrument. The magnetic field can induce small currents in coils of the EM sensor, which can be analyzed to determine a distance and/or angle/orientation between the EM sensor and the EM field generator. Further, a medical instrument can include other types of sensors configured to generate sensor data, such as one or more of any of: a camera, a range sensor, a radar device, a shape sensing fiber, an accelerometer, a gyroscope, a satellite-based positioning sensor (e.g., a global positioning system (GPS)), a radio-frequency transceiver, and so on. In some embodiments, a sensor is positioned on a distal end of a medical instrument, while in other embodiments a sensor is positioned at another location on the medical instrument. In some embodiments, a sensor on a medical instrument can provide sensor data to the control system 140 and the control system 140 can perform one or more localization techniques to determine/track a position and/or an orientation of a medical instrument.

In some embodiments, the medical system 100 may record or otherwise track the runtime data that is generated during a medical procedure. This runtime data may be referred to as system data. For example, the medical system 100 may track or otherwise record the sensor readings (e.g., sensor data) from the instruments (e.g., the scope 120 and the needle 170) in data store 145A (e.g., a computer storage system, such as computer readable memory, database, filesystem, and the like). In addition to sensor data, the medical system 100 can store other types of system data in data store 145. For example, in the context of FIG. 1, the system data can further include time series data of the video images captured by the scope 120, status of the robotic system 110, commanded data from an I/O device(s) (e.g., I/O device(s) 146 discussed below), audio data (e.g., as may be captured by audio capturing devices embedded in the medical system 100, such as microphones on the medical instruments, robotic arms, or elsewhere in the medical system), external (relative to the patient) imaging device (such as RGB cameras, LIDAR imaging sensors, fluoroscope imaging sensors, etc.), and image data from the imaging device 190, and the like.

As shown in FIG. 1, the control system 140 includes a model reconstruction module 141 which may include control circuitry that operates on the system data and the two-dimensional image data stored in the case data store 145 to generate a reconstructed three-dimensional image of an anatomy using two or more two-dimensional images. As is discussed in greater detail below, the model reconstruction module 141 may employ machine learning techniques to generate a three-dimensional volumetric model of an anatomy using a trained network and two or more two-dimensional images.

The term “scope” or “endoscope” are used herein according to their broad and ordinary meanings and can refer to any type of elongate medical instrument having image generating, viewing, and/or capturing functionality and configured to be introduced into any type of organ, cavity, lumen, chamber, and/or space of a body. For example, references herein to scopes or endoscopes can refer to a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), a borescope, and so on.

A scope can comprise a tubular and/or flexible medical instrument that is configured to be inserted into the anatomy of a patient to capture images of the anatomy. In some embodiments, a scope can accommodate wires and/or optical fibers to transfer signals to/from an optical assembly and a distal end of the scope, which can include an imaging device, such as an optical camera. The camera/imaging device can be used to capture images of an internal anatomical space, such as a target calyx/papilla of a kidney. A scope can further be configured to accommodate optical fibers to carry light from proximately-located light sources, such as light-emitting diodes, to the distal end of the scope. The distal end of the scope can include ports for light sources to illuminate an anatomical space when using the camera/imaging device. In some embodiments, the scope is configured to be controlled by a robotic system, such as the robotic system 110. The imaging device can comprise an optical fiber, fiber array, and/or lens. The optical components can move along with the tip of the scope such that movement of the tip of the scope results in changes to the images captured by the imaging device.

A scope can be articulable, such as with respect to at least a distal portion of the scope, so that the scope can be steered within the human anatomy. In some embodiments, a scope is configured to be articulated with, for example, five or six degrees of freedom, including X, Y, Z coordinate movement, as well as pitch, yaw, and roll. A position sensor(s) of the scope can likewise have similar degrees of freedom with respect to the position information they produce/provide. A scope can include telescoping parts, such as an inner leader portion and an outer sheath portion, which can be manipulated to telescopically extend the scope. A scope, in some instances, can comprise a rigid or flexible tube, and can be dimensioned to be passed within an outer sheath, catheter, introducer, or other lumen-type device, or can be used without such devices. In some embodiments, a scope includes a working channel for deploying medical instruments (e.g., lithotripters, basketing devices, forceps, etc.), irrigation, and/or aspiration to an operative region at a distal end of the scope.

The robotic system 110 can be configured to at least partly facilitate execution of a medical procedure. The robotic system 110 can be arranged in a variety of ways depending on the particular procedure. The robotic system 110 can include the one or more robotic arms 112 configured to engage with and/or control the scope 120 to perform a procedure. As shown, each robotic arm 112 can include multiple arm segments coupled to joints, which can provide multiple degrees of movement. In the example of FIG. 1, the robotic system 110 is positioned proximate to the patient's 130 legs and the robotic arms 112 are actuated to engage with and position the scope 120 for access into an access point, such as the urethra of the patient 130. When the robotic system 110 is properly positioned, the scope 120 can be inserted into the patient 130 robotically using the robotic arms 112, manually by the physician 160, or a combination thereof. The robotic arms 112 can also be connected to the EM field generator 180, which can be positioned near a treatment site, such as within proximity to the kidneys of the patient 130.

The robotic system 110 can also include a support structure 114 coupled to the one or more robotic arms 112. The support structure 114 can include control electronics/circuitry, one or more power sources, one or more pneumatics, one or more optical sources, one or more actuators (e.g., motors to move the one or more robotic arms 112), memory/data storage, and/or one or more communication interfaces. In some embodiments, the support structure 114 includes an input/output (I/O) device(s) 116 configured to receive input, such as user input to control the robotic system 110, and/or provide output, such as a graphical user interface (GUI), information regarding the robotic system 110, information regarding a procedure, and so on. The I/O device(s) 116 can include a display, a touchscreen, a touchpad, a projector, a mouse, a keyboard, a microphone, a speaker, etc. In some embodiments, the robotic system 110 is movable (e.g., the support structure 114 includes wheels) so that the robotic system 110 can be positioned in a location that is appropriate or desired for a procedure. In other embodiments, the robotic system 110 is a stationary system. Further, in some embodiments, the robotic system 112 is integrated into the table 150.

The robotic system 110 can be coupled to any component of the medical system 100, such as the control system 140, the table 150, the EM field generator 180, the scope 120, and/or the needle 170. In some embodiments, the robotic system is communicatively coupled to the control system 140. In one example, the robotic system 110 can be configured to receive a control signal from the control system 140 to perform an operation, such as to position a robotic arm 112 in a particular manner, manipulate the scope 120, and so on. In response, the robotic system 110 can control a component of the robotic system 110 to perform the operation. In another example, the robotic system 110 is configured to receive an image from the scope 120 depicting internal anatomy of the patient 130 and/or send the image to the control system 140, which can then be displayed on the display(s) 142. Furthermore, in some embodiments, the robotic system 110 is coupled to a component of the medical system 100, such as the control system 140, in such a manner as to allow for fluids, optics, power, or the like to be received therefrom. Example details of the robotic system 110 are discussed in further detail below in reference to FIG. 12.

The control system 140 can be configured to provide various functionality to assist in performing a medical procedure. In some embodiments, the control system 140 can be coupled to the robotic system 110 and operate in cooperation with the robotic system 110 to perform a medical procedure on the patient 130. For example, the control system 140 can communicate with the robotic system 110 via a wireless or wired connection (e.g., to control the robotic system 110 and/or the scope 120, receive an image(s) captured by the scope 120, etc.), provide fluids to the robotic system 110 via one or more fluid channels, provide power to the robotic system 110 via one or more electrical connections, provide optics to the robotic system 110 via one or more optical fibers or other components, and so on. Further, in some embodiments, the control system 140 can communicate with the needle 170 and/or the scope 170 to receive sensor data from the needle 170 and/or the endoscope 120 (via the robotic system 110 and/or directly from the needle 170 and/or the endoscope 120). Moreover, in some embodiments, the control system 140 can communicate with the table 150 to position the table 150 in a particular orientation or otherwise control the table 150. Further, in some embodiments, the control system 140 can communicate with the EM field generator 180 to control generation of an EM field around the patient 130.

The control system 140 includes various I/O devices configured to assist the physician 160 or others in performing a medical procedure. In this example, the control system 140 includes an I/O device(s) 146 that is employed by the physician 160 or other user to control the scope 120, such as to navigate the scope 120 within the patient 130. For example, the physician 160 can provide input via the I/O device(s) 146 and, in response, the control system 140 can send control signals to the robotic system 110 to manipulate the scope 120. Although the I/O device(s) 146 is illustrated as a controller in the example of FIG. 1, the I/O device(s) 146 can be implemented as a variety of types of I/O devices, such as a touchscreen, a touch pad, a mouse, a keyboard, a surgeon or physician console, virtual reality hardware, augmented hardware, microphone, speakers, haptic devices, and the like.

As also shown in FIG. 1, the control system 140 can include the display(s) 142 to provide various information regarding a procedure. As noted above, the display(s) 142 can present the visualization interface 144 to assist the physician 160 in the percutaneous access procedure (e.g., manipulating the needle 170 towards a target site). The display(s) 142 can also provide (e.g., via the visualization interface 144 and/or another interface) information regarding the scope 120. For example, the control system 140 can receive real-time images that are captured by the scope 120 and display the real-time images via the display(s) 142. Additionally or alternatively, the control system 140 can receive signals (e.g., analog, digital, electrical, acoustic/sonic, pneumatic, tactile, hydraulic, etc.) from a medical monitor and/or a sensor associated with the patient 130, and the display(s) 142 can present information regarding the health or environment of the patient 130. Such information can include information that is displayed via a medical monitor including, for example, a heart rate (e.g., ECG, HRV, etc.), blood pressure/rate, muscle bio-signals (e.g., EMG), body temperature, blood oxygen saturation (e.g., SpO2), CO2, brainwaves (e.g., EEG), environmental and/or local or core body temperature, and so on.

To facilitate the functionality of the control system 140, the control system 140 can include various components (sometimes referred to as “subsystems”). For example, the control system 140 can include control electronics/circuitry, as well as one or more power sources, pneumatics, optical sources, actuators, memory/data storage devices, and/or communication interfaces. In some embodiments, the control system 140 includes control circuitry comprising a computer-based control system that is configured to store executable instructions, that when executed, cause various operations to be implemented. In some embodiments, the control system 140 is movable, such as that shown in FIG. 1, while in other embodiments, the control system 140 is a stationary system. Although various functionality and components are discussed as being implemented by the control system 140, any of this functionality and/or components can be integrated into and/or performed by other systems and/or devices, such as the robotic system 110, the table 150, and/or the EM generator 180 (or even the scope 120 and/or the needle 170). Example details of the control system 140 are discussed in further detail below in reference to FIG. 13.

The imaging device 190 can be configured to capture/generate one or more images of the patient 130 during a procedure, such as one or more x-ray or CT images. In examples, images from the imaging device 190 can be provided in real-time to view anatomy and/or medical instruments, such as the scope 120 and/or the needle 170, within the patient 130 to assist the physician 160 in performing a procedure. The imaging device 190 can be used to perform a fluoroscopy (e.g., with a contrast dye within the patient 130) or another type of imaging technique.

The various components of the medical system 100 can be communicatively coupled to each other over a network, which can include a wireless and/or wired network. Example networks include one or more personal area networks (PANs), local area networks (IANs), wide area networks (WANs), Internet area networks (IANs), cellular networks, the Internet, etc. Further, in some embodiments, the components of the medical system 100 are connected for data communication, fluid/gas exchange, power exchange, and so on, via one or more support cables, tubes, or the like.

Although various techniques and systems are discussed as being implemented as robotically-assisted procedures (e.g., procedures that at least partly use the medical system 100), the techniques and systems can be implemented in other procedures, such as in fully-robotic medical procedures, human-only procedures (e.g., free of robotic systems), and so on. For example, the medical system 100 can be used to perform a procedure without a physician holding/manipulating a medical instrument (e.g., a fully-robotic procedure). That is, medical instruments that are used during a procedure, such as the scope 120 and the needle 170, can each be held/controlled by components of the medical system 100, such as the robotic arm(s) 112 of the robotic system 110.

Three-Dimensional Model Reconstruction Methods and Operations

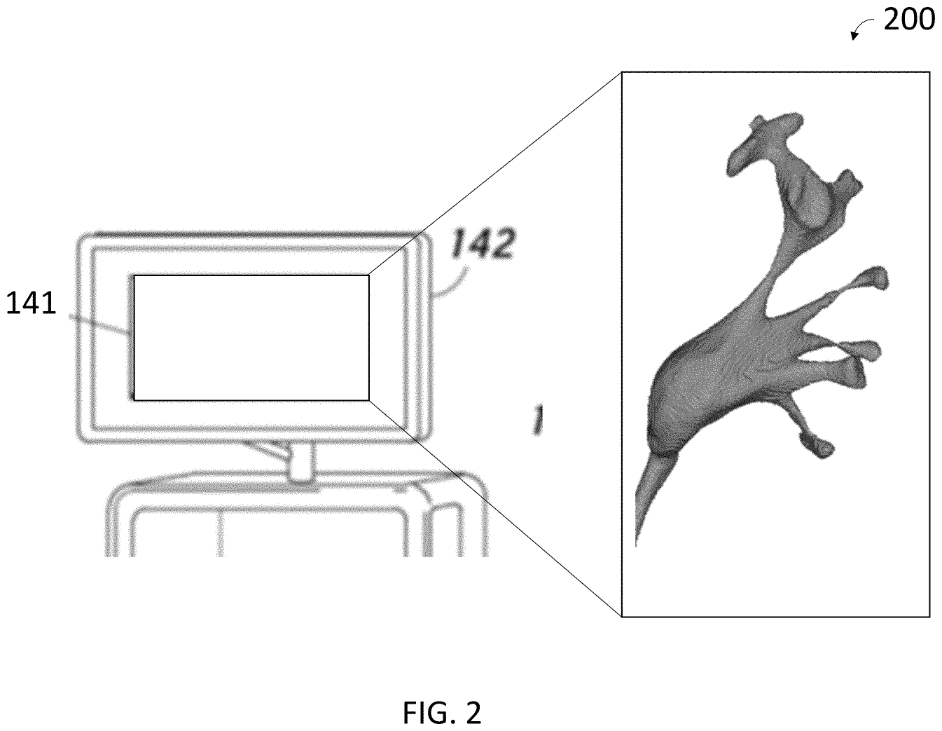

Details of the operations of exemplary model reconstruction systems are now discussed. The methods and operation disclosed herein are described relative to the model reconstruction systems 100 shown in FIG. 1 and the modules and other components shown in FIG. 2 and. However, it is to be appreciated that the methods and operations may be performed by any of the components, alone or in combination, discussed herein.

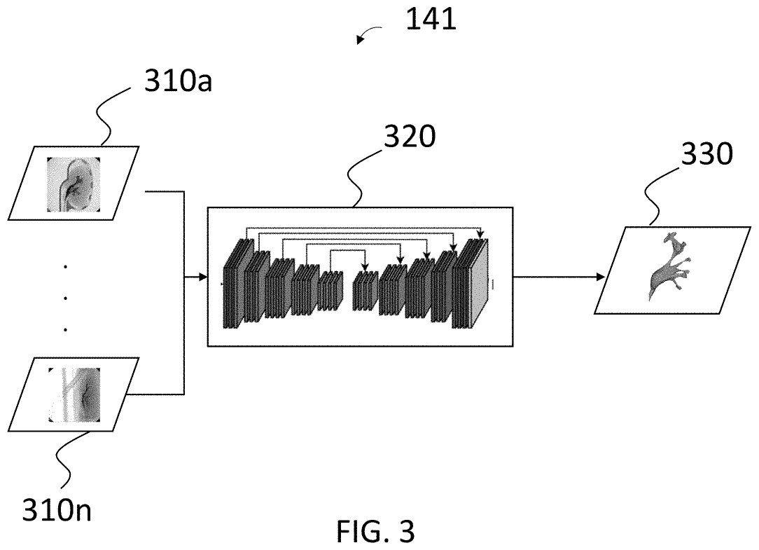

A model reconstruction system may generate a representation of a three-dimensional anatomy based on a comparatively limited number of two-dimensional images acquired during or as part of a medical procedure. FIG. 3 is a block diagram illustrating an example dataflow of the model reconstruction module 141 of FIG. 1, according to an example embodiment. As shown in FIG. 3, the model reconstruction module 141 receives two-dimensional images 310a-n as input and outputs a reconstructed model 330. The reconstruction module 320 may include a neural network that was previously trained on labeled images. The systems, methods, and apparatuses for generated a neural network is described in greater detail below. Some embodiments, additionally or alternatively, may include an engineered or algorithmic solution that processes the two-dimensional images 310a-n to generate the reconstructed model 330 based on identifying or otherwise matching shape priors, calyxes, symantec templates, key points, and the like. A neural network can be trained to automatically recognize key anatomical landmarks like individual calyxes, ureteropelvic junction in two-dimensional images, which will simplify the recognition of the kidney anatomy. An alternative solution is to rely on the clinical knowledge about the most common kidney anatomies. For each anatomy subcategory, kidney examples that belong to this subcategory will be collected. A principal component analysis will be performed on such examples. The principal components generated will capture the main shape variability in the target kidney subcategory. Any linear combination of these components will result in an example of a kidney that belongs to the target subcategory. For a kidney observed in an intra-operative fluoro, an optimal linear combination of principal components will be searched. If such a combination accurately captures the kidney—the kidney belongs the target subcategory. Otherwise it belongs to some other subcategory. The principal component models for each common subcategory will be fitted to the kidney fluoro to recognize to which subcategory belongs the kidney. According to the recognized kidney type, the appropriate neural from module 141 will be selected. Another solution may include a single shot estimation that utilizes semantic connections of the anatomy, such as, in the context of a kidney, the ureteropelvic junction may branch to major calyces, major calyces are connected to minor calyces, and so forth. The reconstructed model 330 may represent the anatomy in a three-dimensional view.

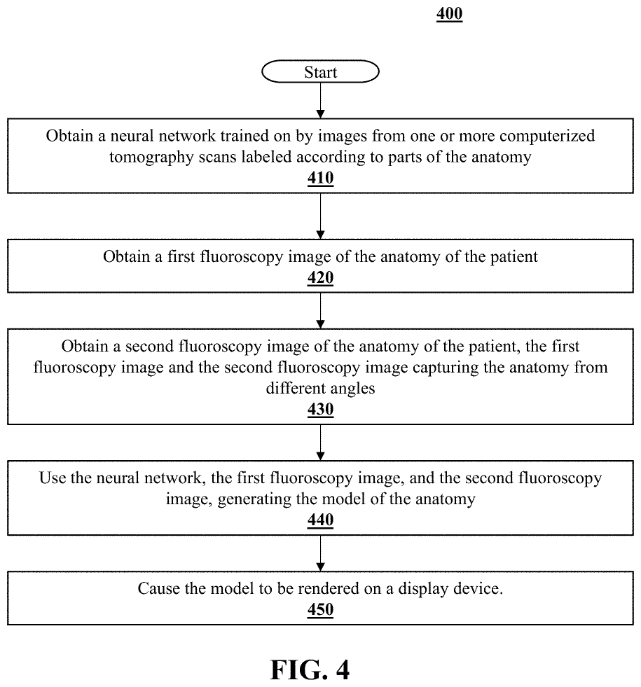

FIG. 4 is a flow-chart illustrating a method 400 to reconstruct a three-dimensional model from two-dimensional images acquired during or as part medical procedure, according to an example embodiment. As FIG. 4 shows, the method 400 may begin at block 410, where the system 100 obtains a neural network trained on by images from one or more computerized tomography scans labeled according to parts of the anatomy. As used herein, a system or apparatus may “obtain” data in any number of mechanisms, such as push or pull models or access through local storage. For example, a neural network service (described in below) may stream the neural network to the model reconstruction module 141 based on determinable events or based on a schedule. In other examples, the model reconstruction module 141 may send a request for the neural network to a neural network service and, in response to the request, the neural network service may send the requested neural network to the reconstruction module 141. Still further, some embodiments may maintain local copies of the neural network and may obtain the neural network via local requests to storage devices.

At block 420, the system 100 may obtain a first fluoroscopy image of the anatomy of the patient. At block 430, the system may obtain a second fluoroscopy image of the anatomy of the patient. The first fluoroscopy image and the second fluoroscopy image may capture images of the same anatomy but from different angles. For example, in some embodiments, the first fluoroscopy image may be acquired from a coronal kidney projection. The coronal kidney projection may offer the best visibility of kidney anatomy. The second fluoroscopy image may be acquired from a sagittal projection (lateral view), as the sagittal projection augments the coronal projection. The angle between the two fluoroscopy images may lie significantly in the interval from 75 to 105 degrees. The fluoroscopic images acquired during blocks 420, 430 may be acquired during the procedure to visual determine a calyx suitable for percutaneous access.

At block 440, the system 100 using the neural network, the first fluoroscopy image, and the second fluoroscopy image, generating the model of the anatomy. In some embodiments, the neural network may include a multi-path recurrent reconstruction neural network. The input to the neural network includes the fluoroscopic images that are passed through two parallel encoder paths. The outputs of two encoder paths are concatenated and then passed through a recurrent unit. The recurrent unit serves to unwrap the two-dimensional input into a three-dimensional volume that will contain the volumetric kidney anatomy reconstructions. The output of the recurrent unit may be passed through a decoder that populates the three-dimensional volume with probabilities to form the kidney anatomy map. The resulting kidney anatomy map passed through a threshold to generate a resulting binary mask of the kidney collective system. A rendering of the kidney collective system (e.g., the model) is the displayed to the user at block 460.

It is to be appreciated that in some embodiments, the input to the neural network may be data derived from the image data rather than the image data itself. For example, the input to neural network can be a skeleton extracted from a pyelogram or it could be key points (calyx, ureteropelvic junction, and the like).

As described above, some embodiments may utilize an engineered solution rather than a neural network. It is to be appreciated that in these embodiments, in the context of the method 400, the engineered or algorithmic solutions will be used rather than the neural network. For example, as described above, one engineered solution may involve a principal component analysis. In this analysis, a three-dimensional volume is reconstructed from the two-dimensional images. It could be vectors of 10 two-dimensional landmarks that define kidney calyces for X samples acquired by the system 100. Or it could be the contours of kidneys in two-dimensional fluoros defined with 100 two-dimensional points. Or it could be surfaces of kidneys defined with 100k three-dimensional points. When a new kidney volume is reconstructed from two dimensional fluoro images, the reconstruction result will be converted into three-dimensional mesh. This mesh will be modeled by most contributive component for each kidney subcategory. If there is subcategory that can model the reconstructed kidney mesh with low error, the kidney is believed to belong to this subcategory. The most contributive components will be used to refine the reconstruction results.

Although the method 400 is discussed in terms of obtaining first and second fluoroscopy images and the neural network is configured to operate on those first and second fluoroscopy images, other embodiments may obtain additional fluoroscopy images. For example, in some cases, it may be desirable to further improve visibility of some calyces and more fluoroscopy images can be acquired at different angles. The multi-path recurrent reconstruction neural network presented above can accommodate additional fluoro images by changing the recurrent unit from a two-to-many architecture, where the input is two fluoroscopy images, to a many-to-many architecture. Thus, these embodiments may allow a user to acquire any number of fluoroscopy images, where each new fluoroscopy image may improve the quality of the resulting reconstruction until the required level of sharpness and accuracy is achieved.

It is to be appreciated that embodiments discussed herein use two-dimensional imaging to reconstruct three-dimensional models of an anatomy without the need to acquire a contrasted computerized tomography scan. Accordingly, embodiments discussed herein may provide a visualization of an anatomy without requiring an additional step or procedure to the workflow and may provide more efficient use of the medical resources and provide a better experience for the patient.

Model Generation Module

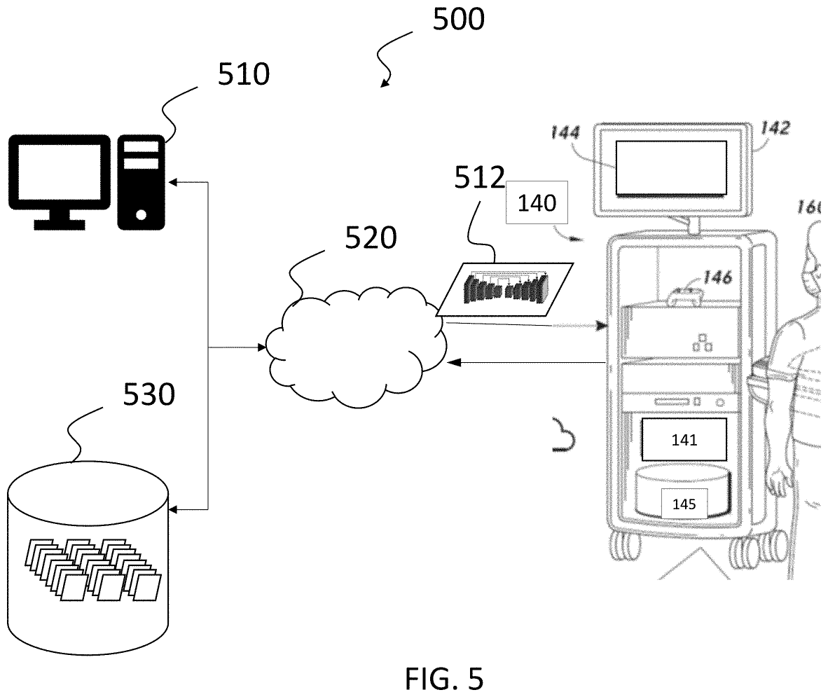

As discussed above, with reference to block 410 of FIG. 4, the system 100 may obtain a neural network trained on by images from one or more computerized tomography scans labeled according to parts of the anatomy. Also discussed above, the neural network may be accessed through a neural network service. The concepts involving the generation of neural network and a neural network service are now described in greater detail. FIG. 5 is a system diagram illustrating a neural network generation system 500, according to an example embodiment. The neural network generation system 500 may include networked communication between the control system 140 of FIG. 1 and a neural network training system 510 via network 520. The network 520 may include any suitable communication network that allows two or more computer systems to communicate with each other, which can include a wireless and/or wired network. Example networks include one or more personal area networks (PANs), local area networks (IANs), wide area networks (WANs), Internet area networks (IANs), cellular networks, the Internet, etc.

The neural network training system 510 may be a computer system configured to generate neural networks 512 usable to generate reconstructed three-dimensional models of an anatomy from two-dimensional images (e.g., fluoroscopy images).

The neural network training system 510 may be coupled to a two-dimensional image database 530. The two-dimensional image database 530 may include annotated images of an anatomy, such as a kidney. For example, contrasted kidney CTs from a pool of patients can be collected and manually annotated by an experienced human observer. As is explained in greater detail below, the images and annotations from the CT scans can be modified to approximate the hardware and software of the medical system receiving the neural network. Further, in some embodiments, the neural network training system 510 may segment the annotated kidney images based on characteristics of the patients or the kidneys shown in the contrasted kidney CTs.

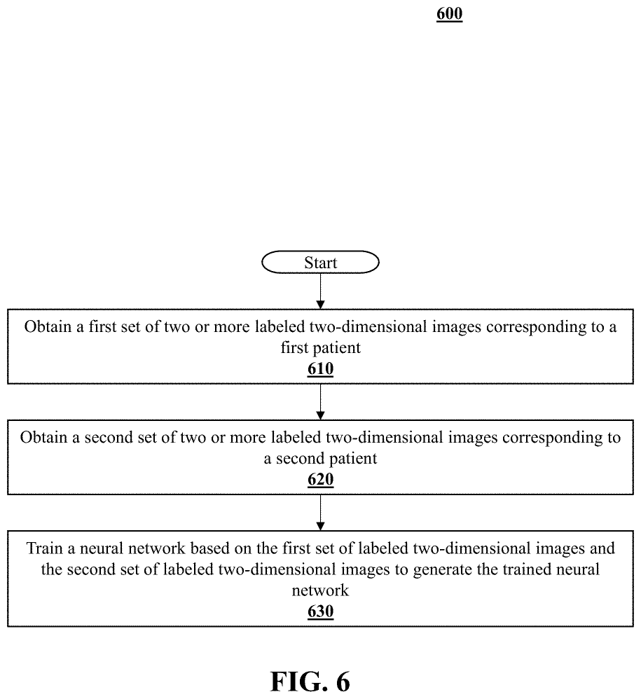

FIG. 6 is a flowchart illustrating a method 600 to generate a trained neural network usable to reconstruct a three-dimensional model from a set of two or more two-dimensional images, according to an example embodiment.

At block 610, the neural network training system 510 obtains a first set of two or more labeled two-dimensional images corresponding to a first patient. The first set of labeled two-dimensional images may be derived from three-dimensional imagery of an anatomy of the first patient. One example of a three-dimensional imagery may be a three-dimensional volumetric output of a CT scan. In this example, the neural network training system 510 may generate the first set of two-dimensional images by creating artificial fluoroscopic images from the three-dimensional volume of a CT scan. In some embodiments, for each CT scan, the neural network training system 510 may generate multiple to many artificial fluoroscopic images from the annotated CT database. By way of example and not limitation, the neural network training system 510 may generate a hundred or hundreds of fluoroscopic images from one orientation (e.g., a coronal orientation) with random oscillation, and a hundred or hundreds of fluoroscopic images from another orientation (e.g., a sagittal orientation) with random oscillation. It is to be appreciated that the random oscillations (say, for example, 15-20 degrees) can account for imperfections of patient positioning during a procedure and to enrich the database of fluoro-CT kidney examples.

It is to be appreciated that the neural network training system 510 may generate the artificial images in any suitable manner. For example, in one embodiment, perform perspective projection of the three-dimensional imagery into a two-dimensional plane. In the context of CT scans, this is one approach for reconstructing radiographs from CT images. Another approach, may be to take a planar slice from the three-dimensional imagery. The slice can then be post-processed to better resemble a two-dimensional image that may be acquired by an imaging device.

At block 620, the neural network training system 510 obtains a second set of two or more labeled two-dimensional images corresponding to a second patient. Similar to block 610, the second set of labeled two-dimensional images may be derived from three-dimensional imagery of the anatomy, such as three-dimensional volumetric output of a CT scan. The neural network training system 510 may generate the second set of two-dimensional images by creating artificial fluoroscopic images from the three-dimensional volume of the CT scan. In some embodiments, for each CT scan, the neural network training system 510 may generate multiple to many artificial fluoroscopic images from the annotated CT database. By way of example and not limitation, the neural network training system 510 may generate a hundred or hundreds of fluoroscopic images from one orientation (e.g., a coronal orientation) with random oscillation, and a hundred or hundreds of fluoroscopic images from another orientation (e.g., a sagittal orientation) with random oscillation.

At block 630, the neural network training system 510 trains a neural network based on the first set of labeled two-dimensional images and the second set of labeled two-dimensional images to generate the trained neural network. As discussed above, the trained neural network may include a multi-path recurrent reconstruction neural network. The trained network may be configured to receive two-dimensional images acquired from a medical system that are then passed through two parallel encoder paths. The trained network is further configured to concatenate the outputs of encoder paths and pass the concatenated output through a recurrent unit. The recurrent unit serves to unwrap the two-dimensional input into a three-dimensional volume that contains the volumetric kidney anatomy reconstructions. The trained neural network is configured to pass the output of the recurrent unit through a decoder that populates the three-dimensional volume with probabilities to form the kidney anatomy map. The resulting map is finally passed through a threshold to generate a binary mask of the kidney collective system.

It is to be appreciated that in some embodiments, the input for training the neural network may be data derived from the image data rather than the image data itself. For example, the input to neural network can be a skeleton extracted from a pyelogram or it could be key points (calyx, ureteropelvic junction, and the like).

At the conclusion of the method 600, in embodiments where the neural network training system 510 is distributed from the control system 140, the neural network training system 510 may send the trained neural network to the control system 140. Once received, the control system 140 may use the trained neural network to reconstruct three-dimensional models of anatomy using two-dimensional images obtained intraoperatively. This use of the trained neural network is discussed above with reference to FIG. 4.

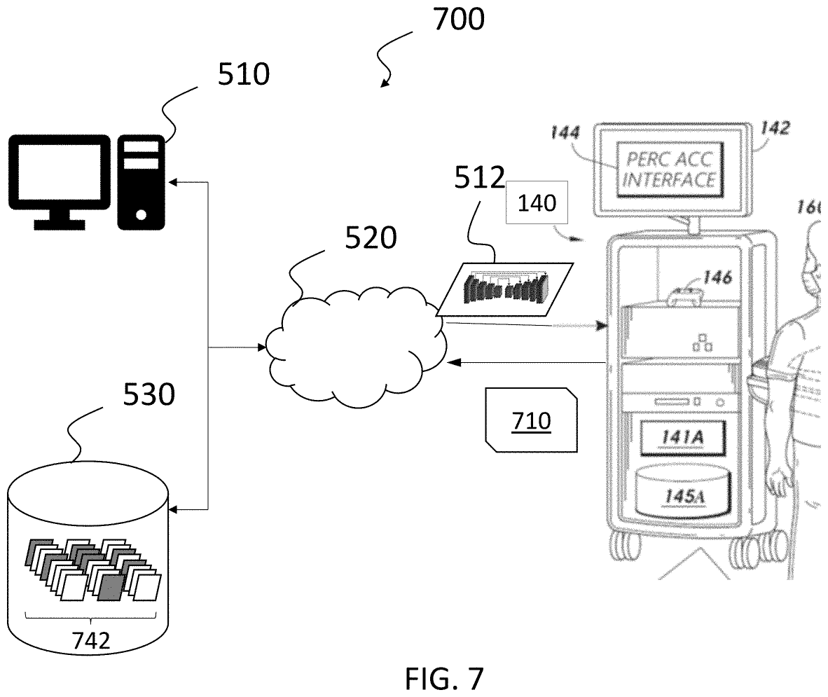

In some embodiments, the neural network generation system 500 may generate trained neural networks that are personalized. As used herein, a personalized trained neural network may refer to a neural network that has been trained in a way that accounts for properties or characteristics of a patient or system that is to use the trained neural network to reconstruct three-dimensional models. FIG. 7 is a system diagram illustrating a personalized neural network generation system 700, according to an example embodiment. The personalized neural network generation system 700 may include many of the components shown and described with reference to the neural network generation system 500 of FIG. 5. The personalized neural network generation system 700 may operate by receiving a request 710 from a control system 140 for a trained neural network. The request 710 may include procedure properties. For example, the procedure properties may include properties of the equipment for the system 100 in which the control system 140 operates. Properties of the equipment may include imaging capabilities of the imaging device (e.g., resolution), dimension and distances between the imaging device and a known location like the bed platform, angles in which the imaging device will be acquiring images, and the like. Additionally or alternatively, the procedure properties may include properties of the patient in which the control system 140 is to perform an operation thereon. Properties of the patient may include age, race, gender, conditions, and the like. In some cases, certain anatomies may have known structures and an identifier of the known structure can be passed as a procedure property. For example, the internal structure of a kidney may have one of four generally known shapes, and the procedure properties that the control system 140 may send to the personalized neural network generation system 700 may include an identification of the type of shape in which to build the trained neural network.

Based on the request 710, the personalized neural network generation system 700 may segment or derive the annotated kidney images based on the procedure properties. In some cases, the annotated kidney images may each include properties that characterize the anatomy of the image, such as age, race, gender, conditions, and the like. The personalized neural network generation system 700 may filter out the annotated kidney images with properties that do not match or are outside an acceptable range compared to the procedure properties in the request 710. For example, the personalized neural network generation system 700 can filter out all the annotated kidney images outside an age range specified by the request 710.

Additionally or alternatively, the images and annotations from the CT scans can be modified to approximate the hardware and software of the medical system receiving the neural network. For example, some embodiments of the personalized neural network generation system 700 may rescale the images and their annotations to an isotropic resolution which approximately matches the image resolution of the imaging device 190. This resolution may be specified in the request 710 as a procedure property or the resolution can be inferred based on an identification of the equipment, such as a lookup table mapping make and models of the imaging device to known resolutions. The personalized neural network generation system 700 may also process the images to match a known distance between the imaging device a known reference point, such as the bed. Further, if the procedure properties identify imaging device angles, the personalized neural network generation system 700 may generate the virtual two-dimensional images using the angles listed in the procedure properties of the request 710.

Once the personalized neural network generation system 700 segments or derives the annotated kidney images based on the procedure properties in the request, the personalized neural network generation system 700 may generate the personalized trained neural network using the segmented and/or derived annotated kidney images. Generating the personalized neural network generation system 700 may be performed using a method similar to the embodiments described with reference to the method 500 of FIG. 5. It is to be appreciated that generating a trained neural network with annotated images that share properties with the patient may result in a neural network that is more accurate with respect to the patient. To that end, in some embodiments, the annotated images may include annotated images obtained from the patient. In this way, images from the patient are used to train the neural network, and in these cases, some embodiments of the personalized neural network generation system 700 may weight the images from the patient more heavily than others when setting the parameters of the neural network.

Feedback and Updating Training

In some embodiments, the control system 140 may provide feedback to a neural network generation system, such as those described in reference to FIGS. 5 and 7. Such feedback may include annotations of the reconstructed three-dimensional model. The annotations of the patient's anatomy can be derived from the neural network itself. The annotations of the patient's anatomy can also be entered by an operator of the control system 140. Other types of feedback that the control system may include a number and corresponding angles of fluoroscopic images taken to achieve a given three-dimensional model and confidence levels of the accuracy of the three-dimensional models, in whole or in given areas within the anatomy.

This level of feedback may be incorporated by a neural network generation system. Some embodiments may use this feedback to correlate the number and angles of fluoroscopic images that results in a desirable outcome. This correlation can be provided to the control system as part of the neural network to assist guiding the control system in taking two-dimensional images with the imaging device 190. This guidance can be instructions and suggestions for the number and angles of the two-dimensional images or the guidance can be automated where the control system automatically controls the imaging device to take the two-dimensional images according to the recommendation.

By way of example and not limitation, the internal structure of a kidney can have one of several known shapes. In situations where the three-dimensional orientation of minor calyxes for a particular shape are difficult to reconstruct using two two-dimensional images, the neural network generation system may determine through a detected correlation that three or more two-dimensional images will result in the neural network in producing a comparatively preferred three-dimensional model. In this case, the neural network generation system may generate a recommendation to the control system to take the three or more two-dimensional images with the imaging device. Additionally or alternatively, the angle for the two-dimensional images may be a factor and the neural network generation system may detect a correlation based on the angle, which is then sent as a guidance to the control system, which may ensure a two-dimensional image or images are acquired from a suitable angle to capture a difficult-to-reconstruct calyx or calyces.

Confidence Levels

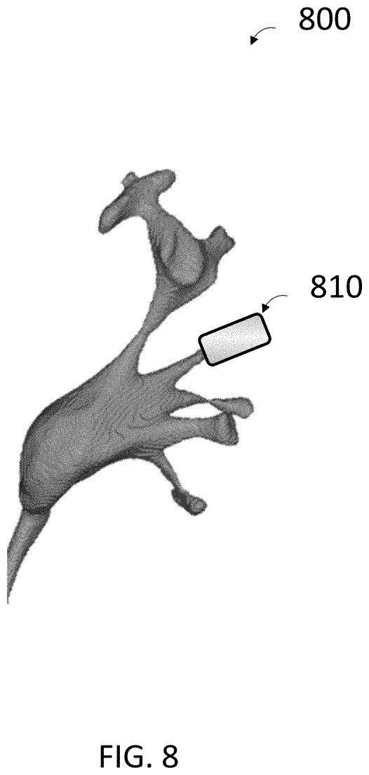

In some embodiments, the neural network may detect a confidence level for the reconstructed three-dimensional model. FIG. 8 is a diagram illustrating a reconstructed three-dimensional model 800, according to an example embodiment. The reconstructed three-dimensional model 800 includes a confidence indicator 810 that is a visual element that indicates a confidence level of the accuracy of a portion of the reconstructed three-dimensional model 800. In the example shown in FIG. 8, the confidence indicator 810 may indicate that the system has a low confidence (as may be measured based on a threshold confidence level) that the portion of the anatomy highlighted by the confidence indicator 810 is accurate. It is to be appreciated that the confidence indicator can be represented as a shading, a pattern, transparency, or the like.

The confidence of the reconstructed anatomy (or a portion thereof) may be determined based on the output of the neural network. For example, a neural network may be trained to reconstruct three-dimensional kidney images in the form of binary masks. In some embodiments, the binary mask may actually be represented by a three-dimensional array with values ranging from 0 to 1. The network may assign values closer to 0.5 to represent less certainty about the corresponding pixels in the three-dimensional array.

Based on the confidence, the system may recommend to the operator to take additional two-dimensional images, possibly at different angles or better-quality images if the was an external condition that lowered the overall quality of the initial two-dimensional images, such as poor disbursement of the contrast agent or poor depth or focus. In some embodiments, the initial confidence levels, remedial actions, and the resulting confidence levels are communicated back to the neural network generation system as feedback that may be used to improve the training data and generate recommendations for future uses.

Implementing Systems and Terminology

Implementations disclosed herein provide systems, methods and apparatus to reconstruct a three-dimensional model of an anatomy using two-dimensional images. Various implementations described herein provide for improved visualization of an anatomy during a medical procedure using a medical robot.

The system 100 can include a variety of other components. For example, the system 100 can include one or more control electronics/circuitry, power sources, pneumatics, optical sources, actuators (e.g., motors to move the robotic arms), memory, and/or communication interfaces (e.g. to communicate with another device). In some embodiments, the memory can store computer-executable instructions that, when executed by the control circuitry, cause the control circuitry to perform any of the operations discussed herein. For example, the memory can store computer-executable instructions that, when executed by the control circuitry, cause the control circuitry to receive input and/or a control signal regarding manipulation of the robotic arms and, in response, control the robotic arms to be positioned in a particular arrangement.

The various components of the system 100 can be electrically and/or communicatively coupled using certain connectivity circuitry/devices/features, which can or may not be part of the control circuitry. For example, the connectivity feature(s) can include one or more printed circuit boards configured to facilitate mounting and/or interconnectivity of at least some of the various components/circuitry of the system 100. In some embodiments, two or more of the control circuitry, the data storage/memory, the communication interface, the power supply unit(s), and/or the input/output (I/O) component(s), can be electrically and/or communicatively coupled to each other.

The term “control circuitry” is used herein according to its broad and ordinary meaning, and can refer to any collection of one or more processors, processing circuitry, processing modules/units, chips, dies (e.g., semiconductor dies including come or more active and/or passive devices and/or connectivity circuitry), microprocessors, micro-controllers, digital signal processors, microcomputers, central processing units, graphics processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry can further comprise one or more, storage devices, which can be embodied in a single memory device, a plurality of memory devices, and/or embedded circuitry of a device. Such data storage can comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware state machine (and/or implements a software state machine), analog circuitry, digital circuitry, and/or logic circuitry, data storage device(s)/register(s) storing any associated operational instructions can be embedded within, or external to, the circuitry comprising the state machine, analog circuitry, digital circuitry, and/or logic circuitry.

The term “memory” is used herein according to its broad and ordinary meaning and can refer to any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data.

Computer-readable media that can be implemented in accordance with embodiments of the present disclosure includes, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is understood with the context as used in general to convey that an item, term, element, etc. may be either X, Y, or Z. Thus, such conjunctive language is not generally intended to imply that certain embodiments require at least one of X, at least one of Y, and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the disclosure should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method to generate a model of an anatomy of a patient, the method comprising:
obtaining a neural network trained on by images from one or more computerized tomography scans labeled according to parts of the anatomy;
obtaining a first fluoroscopy image of the anatomy of the patient;
obtaining a second fluoroscopy image of the anatomy of the patient, the first fluoroscopy image and the second fluoroscopy image capturing the anatomy from different angles;
using the neural network, the first fluoroscopy image, and the second fluoroscopy image, generating the model of the anatomy; and
causing the model to be rendered on a display device.

2. The method of claim 1, further comprising:
obtaining a third fluoroscopy image of the anatomy of the patient, wherein the generating the model of the anatomy further uses the third fluoroscopy image.

3. The method of claim 1, wherein the different angles are substantially orthogonal to each other.

4. The method of claim 1, further comprising generating a confidence level associated with at least a portion of the model relating to a confidence of an accuracy of the portion.

5. The method of claim 4, further comprising causing a representation of the confidence level to be rendered on the display device in conjunction with the model.

6. A method to generate a trained neural network usable to reconstruct a three-dimensional model from a set of two or more two-dimensional images, the method comprising:
obtaining a first set of two or more labeled two-dimensional images corresponding to a first patient;
obtaining a second set of two or more labeled two-dimensional images corresponding to a second patient; and
training a neural network based on the first set of two or more labeled two-dimensional images and the second set of two or more labeled two-dimensional images to generate the trained neural network.

7. The method of claim 6, further comprising obtaining a property of a third patient, wherein the first set of two or more labeled two-dimensional images corresponding to the first patient is obtained based on a comparison between the first patient and the property.

8. The method of claim 7, wherein the second set of two or more labeled two-dimensional images corresponding to the second patient is obtained based on a comparison between the second patient and the property.

9. The method of claim 6, further comprising processing the first set of two or more labeled two-dimensional images based on a property of a medical system.

10. The method of claim 9, wherein the property of the medical system includes at least one of: an imaging capability of an imaging device, a distance between the imaging device and a known location, or an angle of the imaging device.

11. The method of claim 6, further comprising:
obtaining a first set of two or more unlabeled two-dimensional images corresponding to the first patient; and
generating the three-dimensional model using the first set of two or more unlabeled two-dimensional images and the trained neural network.

12. The method of claim 6, wherein the training of the neural network based on the first set of two or more labeled two-dimensional images and the second set of two or more labeled two-dimensional images further comprises:
deriving a first set of properties from the first set of two or more labeled two-dimensional images;
deriving a second set of properties from the second set of two or more labeled two-dimensional images; and
training the neural network using the first set of properties and the second set of properties.

13. The method of claim 12, wherein the first set of properties includes at least one of: skeletons extracted from the first set of two or more labeled two-dimensional images or key points identifying portions of an anatomy.

14. A non-transitory computer readable storage medium to generate a trained neural network usable to reconstruct a three-dimensional model from a set of two or more two-dimensional images, the non-transitory computer readable storage medium having stored thereon instructions that, when executed, cause a processor of a device to at least:
obtain a first set of two or more labeled two-dimensional images corresponding to a first patient;
obtain a second set of two or more labeled two-dimensional images corresponding to a second patient; and
train a neural network based on the first set of two or more labeled two-dimensional images and the second set of two or more labeled two-dimensional images to generate the trained neural network.

15. The non-transitory computer readable storage medium of claim 14, wherein the instructions further cause the processor to perform:
obtain a property of a third patient, wherein the first set of two or more labeled two-dimensional images corresponding to the first patient is obtained based on a comparison between the first patient and the property.

16. The non-transitory computer readable storage medium of claim 15, wherein the second set of two or more labeled two-dimensional images corresponding to the second patient is obtained based on a comparison between the second patient and the property.

17. The non-transitory computer readable storage medium of claim 14, wherein the instructions further cause the processor to perform:
process the first set of two or more labeled two-dimensional images based on a property of a medical system.

18. The non-transitory computer readable storage medium of claim 17, wherein the property of the medical system includes at least one of: an imaging capability of an imaging device, a distance between the imaging device and a known location, or an angle of the imaging device.

19. The non-transitory computer readable storage medium of claim 14, wherein the instructions further cause the processor to perform:
obtain a first set of two or more unlabeled two-dimensional images corresponding to the first patient; and
generate the three-dimensional model using the first set of two or more unlabeled two-dimensional images and the trained neural network.

20. The non-transitory computer readable storage medium of claim 14, wherein train the neural network based on the first set of two or more labeled two-dimensional images and the second set of two or more labeled two-dimensional images further comprises:
derive a first set of properties from the first set of two or more labeled two-dimensional images;
derive a second set of properties from the second set of two or more labeled two-dimensional images;
train the neural network using the first set of properties and the second set of properties; and
wherein the first set of properties includes at least one of: skeletons extracted from the first set of two or more labeled two-dimensional images or key points identifying portions of an anatomy.
